(19) United States
(12) Patent Application Publication
(10) Pub. No.: US 2016/0051616 A1
(43) Pub. Date: Feb. 25, 2016

(54) Joint pain treatment method of use

(71) Applicant: Alan Zurak, Palisaides Park, NJ (US)

(72) Inventor: Alan Zurak, Palisaides Park, NJ (US)

(21) Appl. No.: 14/931,333

(22) Filed: Nov. 3, 2015

Related U.S. Application Data
(63) Continuation of application No. 14/312,667, filed on Jun. 23, 2014.
(60) Provisional application No. 61/839,212, filed on Jun. 25, 2013.

(51) Int. Cl. A61K 36/8962; A61K 36/53; A61K 35/35; A61K 9/00

(52) U.S. Cl. CPC A61K 36/8962; A61K 9/0014; A61K 36/53; A61K 35/35

(57) ABSTRACT

The invention is directed to a homeopathic joint pain treatment composition. The composition comprises an animal fat such as lard mixed with onions and rosemary herb. The onions and rosemary herb may be finely chopped or pureed to give the composition a homogeneous appearance, and provide for the maximum release of their essential oils. The composition is applied to affected areas, such as joints, topically with massage.

### CROSS-REFERENCE TO RELATED APPLICATIONS

This application is a continuation of co-pending U.S. patent application Ser. No. 14/312,667, filed on Jun. 23, 2014, which claimed priority to U.S. Provisional Application No. 61/839212, filed Jun. 25, 2013, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to homeopathic remedies and in particular to a joint pain treatment composition. For those suffering from joint pain, consistent use of pain-killing drugs reduces their effectiveness and leaves the consumer seeking relief elsewhere. Many consumers are also reluctant to use pharmaceutical pain relievers, whether by prescription or over the counter, which may be habit-forming and have side effects. A homeopathic joint pain treatment composition, comprising an animal fat such as lard mixed with onions, rosemary herb, or other natural ingredients would satisfy this demand. The composition is applied to affected joints topically with massage and reduces joint and muscle pain and inflammation. The composition is also capable of hydrating and smoothing rough skin and wrinkles.

### SUMMARY OF THE INVENTION

Accordingly, the invention is directed to a homeopathic joint pain treatment composition. The composition comprises an animal fat such as lard mixed with onions and rosemary herb. The onions and rosemary herb may be finely chopped or pureed to give the composition a homogeneous appearance, and provide for the maximum release of their essential oils. The composition is applied to affected areas, such as joints topically with massage.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWING

No drawing is provided for the disclosed invention, because a drawing is not necessary for the understanding of the invention under 35 U.S.C. §113, 37 C.F.R. §1.81(a), and MPEP §608.02 (III) (B).

### DETAILED DESCRIPTION OF THE INVENTION

Referring now to the invention in more detail, the invention is directed to a homeopathic joint pain treatment composition. The composition comprises an animal fat such as lard mixed with onions and rosemary herb. The onions and rosemary herb may be finely chopped or pureed to give the composition a homogeneous appearance, and provide for the maximum release of their essential oils. The composition is preferably cream like and is applied to affected areas, such as joints, topically with massage.

The composition relieves stiff joints, injured joints, tired, aching joints from strenuous physical activity, such as sports or manual labor, or from being in a cramped or uncomfortable position for an extended period, such as travel on a trans-oceanic airline flight. The composition relieves joint pain, stiffness and inflammation, relieves muscle pain and stiffness, relieves the discomfort caused by gout, improves range of motion, and improves joint flexibility. The composition also relieves dry and calloused skin and reduces the appearance of wrinkles when applied topically to affected areas of skin.

The composition must be prepared in a specific way, using a specific ratio of the ingredients. Multiple variations of the ratio of the ingredients are contemplated.

The first exemplary embodiment of the composition may be prepared with any type of herb from the rosemary family, any type of onion from the onion family, and any type of animal fat. Alternate embodiments, wherein some of the specified ingredients may be omitted, and pharmaceutical or non-pharmaceutical substitute ingredients may or may not be added to replace them, are also contemplated. Such alternate embodiments may be particularly formulated to accommodate those customers who may suffer from allergies to the three specified ingredients. The substitute ingredients may be active ingredients, inert ingredients, or both.

To use the composition, the user squeezes a small amount from its tube, bottle or other container, and gently massages the composition into the skin around the affected joint. After a few minutes of gently massaging the composition into the affected area, it is recommended that the user wrap the affected areas with an elastic bandage to prevent staining of furniture, clothing and bedding. Using a covering is not necessary, but is recommended to prevent stains on items such as furniture, clothing and bedding.

Components and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

1. A method of using a composition comprising an animal fat component, an onion component, and a rosemary herb component wherein said method comprises applying said composition to a joint, said joint being affected by any of the group of stiffness, aching, inflammation, cramping, injury, gout, or limited range of motion.

2. The method of claim 1 further comprising massaging said joint.

3. The method of claim 2 further comprising wrapping said joint.

4. The method of claim 1 wherein said onion component comprises finely chopped onions.

5. The method of claim 4 further comprising massaging said joint.

6. The method of claim 5 further comprising wrapping said joint.

7. The method of claim 1 wherein said onion component comprises pureed onions.

8. The method of claim 7 further comprising massaging said joint.

9. The method of claim 8 further comprising wrapping said joint.

10. The method of claim 1 wherein said rosemary herb component comprises finely chopped rosemary herb.

11. The method of claim 10 further comprising massaging said joint.

12. The method of claim 11 further comprising wrapping said joint.

13. The method of claim 1 wherein said rosemary herb component comprises pureed rosemary herb.

14. The method of claim 13 further comprising massaging said joint.

15. The method of claim 14 further comprising wrapping said joint.

16. The method of claim 1 wherein said animal fat component comprises rendered pork lard.

17. The method of claim 16 further comprising massaging said joint.

18. The method of claim 17 further comprising wrapping said joint.

19. The method of claim 1 wherein said onion component comprises finely chopped onions, said rosemary herb component comprises finely chopped rosemary herb, and said animal fat component comprises rendered pork lard; said method further comprising massaging said joint and wrapping said joint.

20. The method of claim 1 wherein said onion component comprises pureed onions, said rosemary herb component comprises pureed rosemary herb, and said animal fat component comprises rendered pork lard; said method further comprising massaging said joint and wrapping said joint.
